(19) United States
(12) Patent Application Publication
(10) Pub. No.: US 2020/0268354 A1
(43) Pub. Date: Aug. 27, 2020

(54) APPARATUS AND MEDIUM

(71) Applicant: Canon Medical Systems Corporation, Otawara-shi (JP)

(72) Inventors: Minori IZUMI, Shioya-gun (JP); Tomohisa IMAMURA, Nasushiobara (JP)

(73) Assignee: Canon Medical Systems Corporation, Otawara-shi (JP)

(21) Appl. No.: 16/798,510

(22) Filed: Feb. 24, 2020

(30) Foreign Application Priority Data
Feb. 25, 2019 (JP) 2019-031196

(51) Int. Cl. A61B 8/08; A61B 8/00

(52) U.S. Cl. CPC A61B 8/5207; A61B 8/466; A61B 8/0883; A61B 8/5215; A61B 8/0866; A61B 8/488

(57) ABSTRACT

According to one embodiment, an apparatus includes an acquisition unit and a generation unit. The acquisition unit specifies the cycle of pulses in a part that performs periodic motion based on blood flow information in the part. The generation unit rearranges a plurality of two-dimensional images of the part based on the cycle to generate a three-dimensional image.

### CROSS-REFERENCE TO RELATED APPLICATION

This application is based upon and claims the benefit of priority from Japanese Patent Application No. 2019-031196, filed on 2019 Feb. 25; the entire contents of which are incorporated herein by reference.

### FIELD

Embodiments described herein relate generally to an apparatus and a medium.

### BACKGROUND

In recent years, a medical image diagnosis apparatus has been used to examine a subject. The medical image diagnosis apparatus acquires information on the inside of the subject and creates an image based on the information, thereby generating a medical image of the inside of the subject. Examples of the medical image diagnosis apparatus include an X-ray computed tomography (CT) system, magnetic resonance imaging (MRI) equipment, an ultrasound diagnosis apparatus, and the like.

Among those mentioned above, for example, the ultrasound image diagnosis apparatus is often used to examine a fetus because of its non-invasive nature. The ultrasound image diagnosis apparatus is configured to receive reflected signals of ultrasound waves transmitted toward a part to be diagnosed, and generate an ultrasound image related to the part.

When the ultrasound image diagnosis apparatus is used in the examination of a fetus, especially in the case of examining the heart of a fetus, it is often difficult to display ultrasound three-dimensional images in real time since the fetal heart rate is faster than that of adults. For this reason, a technology called spatio-temporal image correlation (STIC) has been developed to acquire and visualize information related to the fetal heart.

Although STIC is a technology that is valuable in the visualization of the fetal heart, it is not very usable to check, for example, blood flow motion in the placenta or the umbilical cord. This is thought to be because the placenta and the umbilical cord do not move as well as the heart, and it is difficult to identify the pulse of blood flow.

### BRIEF DESCRIPTION OF THE DRAWINGS

FIG. 1 is a functional block diagram illustrating the overall configuration of a medical image diagnosis apparatus (ultrasound image diagnosis apparatus) according to an embodiment;

FIG. 2 is a functional block diagram illustrating the internal configuration of a motion-part image generator according to the embodiment;

FIG. 3 is a diagram illustrating examples of medical images generated according to the embodiment; and

FIG. 4 is a flowchart illustrating the operation of generating a medical image of a motion part using STIC according to the embodiment.

### DETAILED DESCRIPTION

In general, according to one embodiment, an apparatus includes an acquisition unit and a generation unit. The acquisition unit specifies the cycle of pulses in a part that performs periodic motion based on blood flow information in the part. The generation unit rearranges a plurality of two-dimensional images of the part based on the cycle to generate a three-dimensional image.

Exemplary embodiments will be described in detail with reference to the drawings. In the following, an ultrasound image diagnosis apparatus will be described as an example; however, the embodiments are applicable to any other apparatuses (modalities).

### [Configuration of Ultrasound Image Diagnosis Apparatus]

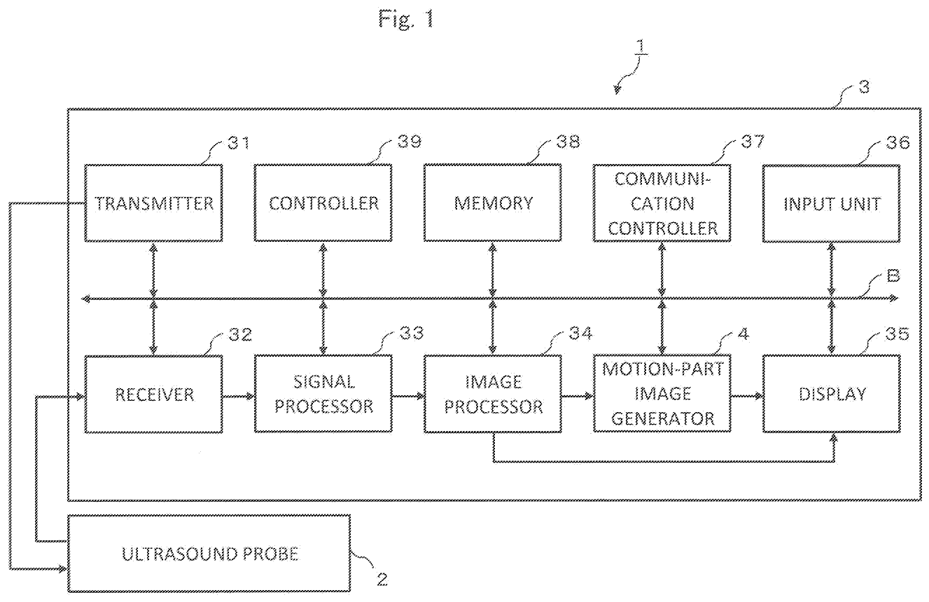

FIG. 1 is a functional block diagram illustrating the overall configuration of an ultrasound image diagnosis apparatus 1 of the embodiment. As illustrated in FIG. 1, the ultrasound image diagnosis apparatus 1 includes an ultrasound probe 2 configured to transmit and receive ultrasound waves to and from a subject, and a main body 3. The ultrasound probe 2 is detachably connected to the main body 3.

The ultrasound image diagnosis apparatus 1 is an example of a medical image diagnosis apparatus that is capable of noninvasively examining a structure inside a subject, the blood flow state, and the like. The ultrasound image diagnosis apparatus 1 is configured to transmit ultrasound waves toward the inside of a subject from the ultrasound probe 2 having transducers (piezoelectric transducers) at its tip, and receive reflected waves caused by acoustic impedance mismatch inside the subject through the transducers of the ultrasound probe 2. The ultrasound image diagnosis apparatus 1 generates ultrasound images based on the received signals.

The ultrasound probe 2 is configured to transmit ultrasound waves into the subject through each of the ultrasound transducers to scan a scan area, and receive reflected waves from the subject as echo signals. The scan includes various types of scans such as, for example, B mode scan and Doppler mode scan. Besides, examples of the ultrasound probe 2 include a sector scan probe, a linear scan probe, a convex scan probe, and the like, and one of them is arbitrarily selected according to the site to be diagnosed.

The main body 3 includes a transmitter 31, a receiver 32, a signal processor 33, an image processor 34, a display 35, and an input unit 36. The transmitter 31 is configured to transmit a drive signal to the ultrasound probe 2. The receiver 32 is configured to receive echo signals from the ultrasound probe 2. The signal processor 33 is configured to process the echo signals. The image processor 34 is configured to generate an ultrasound image. The display 35 is configured to display the ultrasound image. The input unit 36 is configured to receive an input signal as being operated by the user such as an examiner.

The main body 3 further includes a communication controller 37 configured to control the exchange of signals with other devices (not illustrated), a memory 38, and a controller 39 configured to control each part. These parts are connected to a bus B so that they can exchange various signals. The functions of each of the parts are described below in further detail.

Under the control of the controller 39, the transmitter 31 generates a drive signal for causing the ultrasound probe 2 to generate ultrasound waves, i.e., an electric pulse signal (hereinafter referred to as “drive pulse”) to be applied to each of the piezoelectric transducers. The transmitter 31 transmits the drive pulse to the ultrasound probe 2. The transmitter 31 includes circuits such as, for example, a reference pulse generator, a delay controller, a drive pulse generator, and the like (not illustrated), and those circuits perform the functions mentioned above.

The receiver 32 receives an echo signal (received signal from the ultrasound probe 2). The receiver 32 performs phasing addition on the received signal, and outputs the resultant signal to the signal processor 33. The receiver 32 may receive various signals such as color Doppler signals as well as echo signals mentioned above.

The signal processor 33 generates various types of data using the received signal from the ultrasound probe 2 fed by the receiver 32, and outputs the data to the image processor 34 and the controller 39. The signal processor 33 includes, for example, a B mode processing circuit (or a Bc mode processing circuit), a Doppler mode processing circuit, a color Doppler mode processing circuit, and the like (not illustrated). The B mode processing circuit visualizes amplitude information of the received signal, and generates data based on a B mode signal.

The Doppler mode processing circuit extracts Doppler shift frequency components from the received signal, and applies fast Fourier transform (FFT) or the like thereto, thereby generating Doppler signal data of blood flow information. The color Doppler mode processing circuit visualizes the blood flow information based on the received signal, and generates data based on a color Doppler mode signal. In this manner, the color Doppler mode processing circuit extracts power components, velocity components, and variance values from the received signal over multiple points in a two-dimensional or three-dimensional space as information on blood flow in motion, thereby generating Doppler signal data.

The signal processor 33 may include a processing circuit that visualizes the blood flow at a high frame rate. In this case, the processing circuit performs ultrasound scan based on a known method. For example, the processing circuit applies averaging to a plurality of received signals of each scan line obtained by transmitting/receiving ultrasound waves a plurality of times with respect to each scan line. Alternatively, the processing circuit performs low-pass filtering similar to averaging to obtain received signals of each of scan lines in a part to be scanned, and performs high-pass filtering on the received signals in the frame direction, thereby acquiring information on the motion of the part (high frame-rate method). Reference may be had to, for example, Japanese Patent No. 6104749.

The image processor 34 generates two-dimensional or three-dimensional ultrasound images related to the scan area based on the data supplied from the signal processor 33. For example, the image processor 34 generates volume data related to the scan area based on the data supplied. Then, from the volume data generated, the image processor 34 generates data of a two-dimensional ultrasound image by multi-planar reconstruction (MPR) or data of a three-dimensional ultrasound image by volume rendering. The image processor 34 outputs the two-dimensional or three-dimensional ultrasound image to the display 35. Examples of the ultrasound image include a B mode image, a Doppler mode image, a color Doppler mode image, an M mode image, and the like.

The display 35 displays various images such as the ultrasound image generated by the image processor 34 and an operation screen (e.g., graphical user interface (GUI) configured to receive various instructions from the user) under the control of the controller 39. The display 35 is also capable of displaying the electrocardiogram of a subject to be examined together with the ultrasound image. As the display 35, for example, a liquid crystal display (LCD), an organic electroluminescence (EL) display, or the like can be used.

The input unit 36 receives various input operations made by the user to provide, for example, an instruction to display an image or switch images, designation of mode, various settings, and the like. For example, input devices such as buttons, a keyboard, a trackball, GUI, a touch panel displayed on the display 35, or the like can be used as the input unit 36.

The input unit 36 may include, for example, an input device used to perform STIC processing (described later), or an input device used to select a part to be subjected to the STIC processing, such as the fetal heart, and the blood flow in the placenta or the umbilical.

Incidentally, in this embodiment, the display 35 and the input unit 36 are each described as one constituent element of the ultrasound image diagnosis apparatus 1 as illustrated in FIG. 1; however, it is not so limited. The display 35 need not necessarily be a constituent element of the ultrasound image diagnosis apparatus 1, and may be separated therefrom as a separate display. The input unit 36 may be a touch panel displayed on the separate display.

The communication controller 37 enables the ultrasound image diagnosis apparatus 1 to communicate with, for example, medical image diagnosis apparatuses (modalities), servers, workstations, and the like (not illustrated) each connected to a communication network (not illustrated). Information and medical images exchanged between the communication controller 37 and other devices via the communication network may be in conformity with any standard such as digital imaging and communication in medicine (DTCOM) or the like. The connection to the communication network or the like may be either wired or wireless.

The memory 38 is formed of, for example, a semiconductor memory or a magnetic disk. The memory 38 stores programs to be executed by the controller 39 and data.

The controller 39 comprehensively controls each part of the ultrasound image diagnosis apparatus 1. The controller 39 causes the display 35 to display the ultrasound image generated by the image processor 34. Besides, the controller 39 causes a motion-part image generator 4 (described later) to perform processes for generating a three-dimensional image of the blood flow in the placenta or the umbilical cord.

The motion-part image generator 4 generates a medical image related to a part that performs periodic motion (cyclically repeated motion). Examples of the part that performs periodic motion include the fetal heart, the blood vessels (blood flow) of the placenta and the umbilical cord. The motion-part image generator 4 performs STIC processing to generate a three-dimensional image related to the fetal heart, a three-dimensional image illustrating the blood flow in the blood vessels of the placenta and the umbilical cord, or the like.

The STIC processing is performed in the following procedure. First, a part that performs periodic motion is scanned at a low speed using the ultrasound probe 2 to capture a two-dimensional image related to the part. Since the ultrasound probe 2 is moved at a low speed, frame data acquired by a single scan includes a plurality of frames of pulses.

Next, the cycles (periodicity) of pulses are specified in the frame data acquired. For example, when the fetal heart is scanned, the heartbeats of the fetus are detected, and the cycles are specified. Then, images are sorted according to the phases of the cycles specified, and rearranged in time order such as, for example, from end-systole to end-diastole.

Then, images of the same phase are combined to generate a three-dimensional image. By moving the three-dimensional image thus generated along the time axis, one pulse in the target region can be displayed as a four-dimensional image.

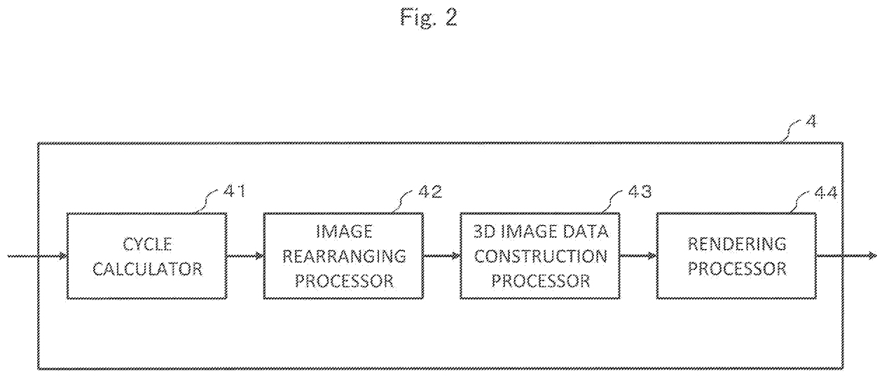

FIG. 2 is a functional block diagram illustrating the internal configuration of the motion-part image generator 4 of the embodiment. The motion-part image generator 4 includes a cycle calculator 41, an image rearranging processor 42, a three-dimensional (3D) image data construction processor 43, and a rendering processor 44.

The cycle calculator 41 calculates the cycle of pulses in a part that performs periodic motion. The data used for the calculation varies depending on the target subjected to the STIC processing. Specifically, when the target is the fetal heart, the cycle calculator 41 specifies the pulses (heartbeats) using a B-mode echo signal.

Besides, in this embodiment, when the target is the blood flow in the placenta or the umbilical cord, information on the blood flow (blood flow information) is used. Specifically, the cycle calculator 41 specifies the cycle of pulses using a color Doppler signal as the blood flow information (fluid information). As described above, while three types of components, i.e., power components, velocity components, and variance values, can be extracted as color Doppler signals, power components are used in this example.

Differently from the case of specifying heartbeats, echo signals are not used when the target is the blood flow in the placenta or the umbilical cord. This is because the placenta and the umbilical cord move much less than the heart, and therefore the required cycle cannot be specified based on echo signals. Since the blood vessels of the placenta and the umbilical cord move more than the placenta and the umbilical cord, the blood flow information can be used to specify the periodicity of pulses therein.

The algorithm for calculating the cycle of pulses does not vary depending on the target, and the same known algorithm is used. Specifically, for example, an FFT is performed in the time axis direction at a certain point in the blood vessel of the placenta or the umbilical cord to specify the cycle at that point.

The image rearranging processor 42 sorts images according to the phase at which they were acquired and rearranges them based on the cycle specified by the cycle calculator 41. For example, the image rearranging processor 42 extracts images at the same time phase using an autocorrelation function.

The three-dimensional image data construction processor 43 generates three-dimensional image data based on two-dimensional images at the same time phase extracted by the image rearranging processor 42. In FIG. 2, the three-dimensional image data construction processor 43 is indicated as “3D image data construction processor”.

The rendering processor 44 performs image processing on the three-dimensional image data generated by the three-dimensional image data construction processor 43 to generate a display image, and sends the display image to the display 35. By a series of these processes, a medical image related to a part that performs periodic motion is generated and displayed.

For example, the image processor 34 can perform the processing of the rendering processor 44. In this case, the three-dimensional image data construction processor 43 sends the three-dimensional image data to the image processor 34.

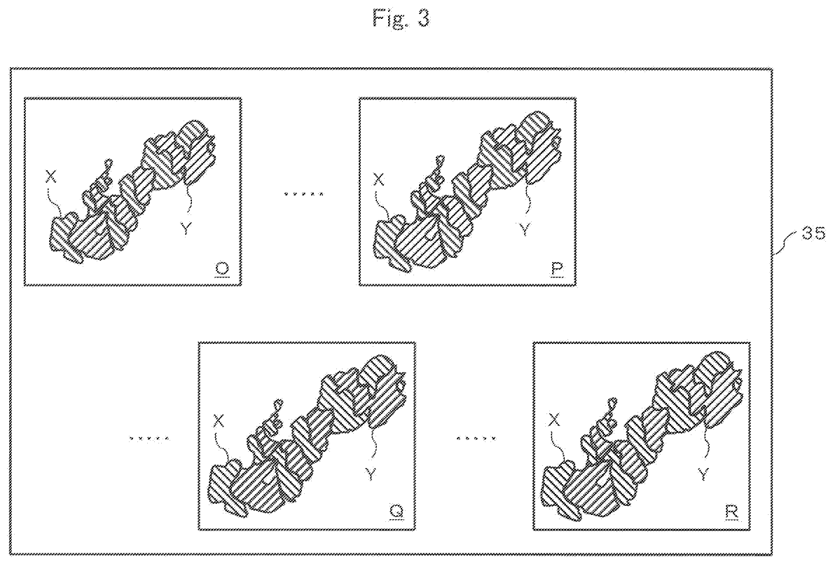

FIG. 3 is a diagram illustrating examples of medical images generated according to the embodiment. In FIG. 3, each of four images illustrates the blood flow in the umbilical blood vessels, among the blood vessels of the placenta and the umbilical cord that have been subjected to the STIC processing. The four images are part of STIC images generated based on images captured from end-systole to end-diastole.

As illustrated in FIG. 3, the screen of the display 35 is divided into upper and lower sections, and the images are displayed such that two of them are placed in each section. On the display 35, an upper left image O illustrates the umbilical blood vessels at end-systole, while a lower right image R illustrates those at the end of end-diastole. That is, the image O, an upper right image P, a lower left image Q, and the image R illustrate, in this order, the periodic motion of the umbilical blood vessels. In this example, the images O to R are displayed in frames of the same size.

Each of the images O to R illustrates areas hatched in two different directions: upper left to lower right; and lower left to upper right. This is to distinguish the blood flow from the womb to the fetus and the blood flow from the fetus to the womb in the umbilical cord.

In an actual STIC image, areas in the umbilical cord where the blood flows from the womb to the fetus are displayed in red, while areas where the blood flows from the fetus to the womb are displayed in blue. In the example of FIG. 3, areas X represent the blood flow from the womb to the fetus. On the other hand, areas Y represent the blood flow from the fetus to the womb.

A plurality of dots are illustrated between the images (between the images O and P, the images P and Q, the images Q and R). The dots represent a plurality of STIC images, which are not illustrated in FIG. 3, but are supposed to be there.

Referring to FIG. 3, the STIC images O to R each illustrate the umbilical blood vessels having a plurality of areas X and areas Y. Comparing the image O that illustrates end-systolic blood flow and the image R that illustrates end-diastolic blood flow, the blood vessels illustrated in the image R are larger than those illustrated in the image O.

That is, the images O to R are displayed such that the size of the umbilical blood vessels gradually increases in the image frames from the image O to the image R along with their expansion. Accordingly, it can be seen that the umbilical blood vessels expand as a whole from end-systole to end-diastole.

Incidentally, upon displaying images that illustrates the blood flow in the blood vessels of the placenta or the umbilical cord, it is possible to freely set the number of pulses for which image data is to be displayed. In the case of displaying a sequence of images from end-diastole to end-systole, it can be observed how the blood vessels of the placenta or the umbilical cord contract.

As described above, in the STIC processing, the cycles of pulses in the blood vessels of the placenta or the umbilical cord are specified by using the power components of color Doppler signals. With this, very clear images with little blur can be obtained. Further, the images are displayed along the time axis of the cycles, which makes it possible to observe the motion of the blood vessels of the placenta or the umbilical cord. The images can also be displayed as a moving image along the time axis of the cycles.

Although four images are illustrated as being arranged in upper and lower sections in FIG. 3, the illustration is by way of example only. The arrangement and the number of images displayed on the display 35 are not limited as illustrated but can be arbitrarily set. Besides, various other items that are not illustrated in FIG. 3 may be displayed on the display 35.

In this embodiment, the motion-part image generator 4 performs the STIC processing; however, the controller 39 may implement the motion-part image generator 4 as a motion-part image generation function.

In this case, for example, the motion-part image generation function of the controller 39 can be realized by a computer program that is executed by a processor and stored in a predetermined memory, the memory 38, or the like. The term “processor” as used herein refers to a circuit such as, for example, a dedicated or general central processing unit (CPU) arithmetic circuit (circuitry), an application specific integrated circuit (ASIC), a programmable logic device such as a simple programmable logic device (SPLD) and a complex programmable logic device (CPLD), a field programmable gate array (FPGA), or the like.

The processor reads out, for example, a program stored in the memory 38 or directly incorporated in the circuit of the processor and executes it, thereby realizing the function. Each processor may be provided with a recording circuit for storing the program. The recording circuit may also store, for example, a program corresponding to the functions of the signal processor 33 illustrated in FIG. 1, and may have the configuration of the memory 38 illustrated in FIG. 1. The memory is formed of a storage device, examples of which include a semiconductor memory and a magnetic disk such as a general random access memory (RAM) and a hard disc drive (HDD).

A description has been given of the functions of the parts that implement the operation of the ultrasound image diagnosis apparatus 1. Each of the parts can be configured by a circuit. For example, the image processor may be an image processing circuit, the motion-part image generator may be a motion-part image generation circuit, and the controller may be a control circuit.

### [Operation]

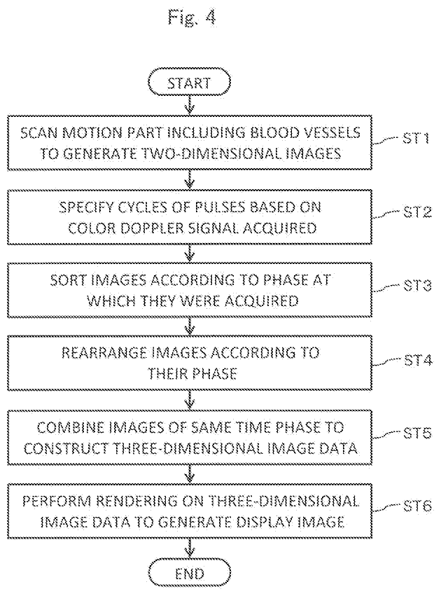

Next, with reference to FIG. 4, a description will be given of the process of acquiring a three-dimensional image of the blood flow in the placenta or the umbilical cord. FIG. 4 is a flowchart illustrating the operation of generating a medical image of a motion part using STIC according to the embodiment.

Incidentally, in order to generate an image based on STIC instead of generating a normal ultrasound image, the examiner is required to provide an instruction to that effect through the input unit 36 so that a signal indicating the instruction is sent to the controller 39. The information used in the STIC processing varies depending on the target, which may be the heart of the fetus, or the blood flow in the placenta or the umbilical cord. Accordingly, it is assumed herein that an instruction signal to perform the STIC processing for displaying the blood flow in the placenta or the umbilical cord has already been sent to the controller 39 through the input unit 36 in response to an instruction from the examiner.

First, the examiner scans a motion part including the blood vessels of the placenta or the umbilical cord with the ultrasound probe 2. In the ultrasound image diagnosis apparatus 1, the signal processor 33 and the image processor 34 generate two-dimensional images based on the scan result (ST1). The two-dimensional images are sent to the motion-part image generator 4. In addition, a color Doppler signal received by the ultrasound probe 2 is also sent to the motion-part image generator 4 via the signal processor 33 and the image processor 34.

In the motion-part image generator 4, first, the cycle calculator 41 specifies the cycles of pulses in the blood vessels based on, for example, the power component of the color Doppler signal (ST2). The cycle calculator 41 sends information on the cycles of pulses in the blood vessels to the image rearranging processor 42.

The image rearranging processor 42 sorts the two-dimensional images based on the information on the cycles of pulses in the blood vessels (ST3). The image rearranging processor 42 then rearranges the two-dimensional images according to their phase (ST4).

The three-dimensional image data construction processor 43 combines two-dimensional images of the same time phase and constructs three-dimensional image data with respect to each time phase (ST5). Thereafter, the rendering processor 44 performs predetermined rendering on the three-dimensional image data, thereby generating a display image (ST6). The display image is sent to the display 35 and displayed, for example, as illustrated in FIG. 3.

In this manner, the cycles of pulses are specified based on the power component of the color Doppler signal in STIC processing for displaying a three-dimensional image that illustrates the blood flow in the placenta or the umbilical cord. With this, the periodicity can be specified with accuracy. Further, the blood flow in the blood vessels of the placenta and the umbilical cord that have less motion can be clearly displayed.

In addition, by combining the STIC processing in which the cycles of pulses in blood vessels are specified based on the power component of the color Doppler signal and the high frame-rate method described above, the motion of blood vessels in a part that moves less can be displayed reliably. Moreover, even finer blood vessels can be displayed with high definition.

According to at least one embodiment described above, the periodicity of pulses in a target can be specified with accuracy in STIC processing.

In the above embodiment, the ultrasound image diagnosis apparatus 1 is described as including the ultrasound probe 2; however, the ultrasound image diagnosis apparatus 1 need not necessarily include an ultrasound probe. Besides, an ultrasound probe can be configured to have all the constituent elements and functions of the ultrasound image diagnosis apparatus 1 and used as an ultrasound image diagnosis apparatus. In this case, STIC images related to the blood flow in the blood vessels of the placenta or the umbilical cord generated by the ultrasound probe are displayed on a separate display device such as a tablet or the like.

By connecting an ultrasound probe to a general-purpose device such as a personal computer, the above-described processing can be performed on the device to generate STI images. The device can be portable or stationary. Examples of the device include various types of apparatuses such as stand-alone ultrasound image diagnosis apparatuses and those used for research and development purposes.

While certain embodiments have been described, these embodiments have been presented by way of example only, and are not intended to limit the scope of the inventions. Indeed, the novel embodiments described herein may be embodied in a variety of other forms; further, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An apparatus comprising processing circuitry configured to:
specify a cycle of pulses in a part that performs periodic motion based on blood flow information in the part; and
rearrange a plurality of two-dimensional images of the part based on the cycle to generate a three-dimensional image.

2. The apparatus of claim 1, wherein
the blood flow information includes a Doppler signal, and
the processing circuitry is further configured to specify the cycle of pulses based on the Doppler signal or a B-mode echo signal depending on the part.

3. The apparatus of claim 1, wherein the processing circuitry is further configured to specify the cycle of pulses based on the blood flow information when the part is a blood vessel of a placenta or an umbilical cord.

4. The apparatus of claim 2, wherein the processing circuitry is further configured to specify the cycle of pulses based on the Doppler signal when the part is a blood vessel of a placenta or an umbilical cord.

5. The apparatus of claim 1, wherein the processing circuitry is further configured to specify the cycle of pulses based on a B-mode echo signal when the part is a heart.

6. The apparatus of claim 5, wherein the processing circuitry is further configured to specify the cycle of pulses based on the echo signal when the part is a heart.

7. The apparatus of claim 1, wherein the blood flow information is a power component of a color Doppler signal.

8. The apparatus of claim 1, wherein the blood flow information is a velocity component or a variance value of a color Doppler signal.

9. The apparatus of claim 1, wherein the processing circuitry further configured to performs ultrasound scan based on a method in which
a received signal is obtained with respect to each of scan lines in the part by
applying averaging to a plurality of received signals of the scan lines obtained by transmitting and receiving ultrasound waves a plurality of times with respect to each of the scan lines, or
performing low-pass filtering similar to the averaging, and
high-pass filtering is performed on the received signal in a frame direction to acquire information on motion of the part.

10. The apparatus of claim 1, wherein the processing circuitry further configured to generate a plurality of three-dimensional images in the cycle by using a plurality of two-dimensional images of different places in the part acquired at the same time phase in the cycle.

11. A medium having computer-readable program codes that, when executed, cause a computer to:
specify a cycle of pulses in a part that performs periodic motion based on blood flow information in the part; and
rearrange a plurality of two-dimensional images of the part based on the cycle to generate a three-dimensional image.

12. An apparatus comprising processing circuitry configured to:
specify a cycle of pulses in a part that performs periodic motion based on fluid information in the part; and
rearrange a plurality of two-dimensional images of the part based on the cycle to generate a three-dimensional image.
